(19) United States
(12) Patent Application Publication
(10) Pub. No.: US 2016/0313296 A1
(43) Pub. Date: Oct. 27, 2016

(54) USE OF ORP FOR CHARACTERIZING STROKE PATIENTS

(71) Applicant: Aytu BioScience, Inc., Englewood, CO (US)

(72) Inventors: Kim Bjugstad, Lonetree, CO (US); Raphael Bar-Or, Denver, CO (US); David Bar-Or, Englewood, CO (US)

(21) Appl. No.: 15/135,222

(22) Filed: Apr. 21, 2016

Related U.S. Application Data
(60) Provisional application No. 62/150,720, filed on Apr. 21, 2015.

(51) Int. Cl. G01N 33/487

(52) U.S. Cl. CPC G01N 33/48707; G01N 2800/2871; G01N 2800/56

(57) ABSTRACT

Methods and systems for measuring and using the oxidation-reduction potential (ORP) of a biological sample are provided. Also provided are methods of characterizing an individual who has suffered a stroke by measuring the ORP of a biological sample. The disclosed methods can be used to characterize the individual with regard to their likelihood of survival, severity of the stroke and their estimated length of stay in a medical facility.

### CROSS-REFERENCE TO RELATED APPLICATIONS

This application claims the benefit of U.S. Provisional Application No. 62/150,720, filed 21 Apr. 2015, the entirety of which is hereby incorporated by reference.

### TECHNICAL FIELD

The present invention relates to methods and apparatuses for measuring the oxidation-reduction potential of a fluid sample and methods of using the same to monitor stroke patients.

### BACKGROUND OF INVENTION

Whole blood and blood products, such as plasma and serum, have oxidation- reduction potentials (ORP). Clinically the ORP of blood, plasma and serum provides the oxidative status of an animal. More particularly, the ORP of blood, plasma and serum is related to health and disease.

An oxidation-reduction system, or redox system, involves the transfer of electrons from a reductant to an oxidant according to the following equation:

oxidant+ne⁻ [symbol] reductant (1)

where ne⁻ equals the number of electrons transferred. At equilibrium, the redox potential

(E), or oxidation-reduction potential (ORP), is calculated according to the Nernst-Peters equation:

E(ORP)=Eₒ−RT/nF In [reductant]/[oxidant] (2)

where R (gas constant), T (temperature in degrees Kelvin) and F (Faraday constant) are constants. Eₒ is the standard potential of a redox system measured with respect to a hydrogen electrode, which is arbitrarily assigned an Eₒ of 0 volts, and n is the number of electrons transferred. Therefore, ORP is dependent on the total concentrations of reductants and oxidants, and ORP is an integrated measure of the balance between total oxidants and reductants in a particular system. As such, ORP provides a measure of the overall oxidative status of a body fluid or tissue of a patient.

Oxidative stress is caused by a higher production of reactive oxygen and reactive nitrogen species or a decrease in endogenous protective antioxidative capacity. Oxidative stress has been related to various diseases and aging, and it has been found to occur in all types of critical illnesses. See, e.g., Veglia et al., Biomarkers, 11(6): 562-573 (2006); Roth et al., Current Opinion in Clinical Nutrition and Metabolic Care, 7:161-168 (2004); U.S. Pat. No. 5,290,519 and U.S. Patent Publication No. 2005/0142613. Several investigations have shown a close association between the oxidative status of a critically ill patient and the patient's outcome. See Roth et al., Current Opinion in Clinical Nutrition and Metabolic Care, 7:161-168 (2004).

Oxidative stress in patients has been evaluated by measuring various individual markers. See, e.g., Veglia et al., Biomarkers, 11(6): 562-573 (2006); Roth et al., Current Opinion in Clinical Nutrition and Metabolic Care, 7:161-168 (2004); U.S. Pat. No. 5,290,519 and U.S. Patent Publication No. 2005/0142613. However, such measurements are often unreliable and provide conflicting and variable measurements of the oxidative status of a patient. See Veglia et al., Biomarkers, 11(6): 562-573 (2006); Roth et al., Current Opinion in Clinical Nutrition and Metabolic Care, 7:161-168 (2004). The measurement of multiple markers which are then used to provide a score or other assessment of the overall oxidative status of a patient has been developed to overcome the problems of using measurements of single markers. See Veglia et al., Biomarkers, 11(6): 562-573 (2006); Roth et al., Current Opinion in Clinical Nutrition and Metabolic Care, 7:161-168 (2004). Although such approaches are more reliable and sensitive than measurements of a single marker, they are complex and time consuming. Thus, there is a need for a simpler and faster method for reliably measuring the overall oxidative status of a patient.

The oxidation/reduction potential can be measured electrochemically. Electrochemical devices for measuring ORP of blood and blood products typically require large sample volumes (that is, ten to hundreds of milliliters) and long equilibrium periods. Furthermore, the electrochemical devices have large, bulky electrodes that require cleaning between sample measurements. Such electrochemical devices are poorly suited for routine clinical diagnostic testing. It has been suggested to use electrodes that have undergone treatment to prevent biofouling. However, such devices necessarily involve complex manufacturing techniques. Moreover, conventional electrochemical devices have not provided a format that is convenient for use in a clinical setting.

The oxidative and radical characteristics of human blood plasma and its blood components (such as low density lipoproteins, serum albumin, and amino acids) can also be determined from photo chemiluminescence, with and without thermo-initiated free radical generation. A photo chemiluminescent system generally includes a free radical generator and a detector that measures chemiluminometric changes in the presence of an antioxidant. More specifically, the blood plasma sample (or one of its components) containing an amount of antioxidant is contacted and reacted with a known amount of free radicals. The free radicals remaining after contacting the blood plasma sample are determined chemiluminometrically. This type of measurement and detection system is not suitable for rapid, large scale measurements of blood plasma samples in a clinical setting for assessing or monitoring human or animal health.

There remains a need for improved methods and devices for measuring the oxidation-reduction characteristics of biological samples. Further, there is a need for use of such improved methods and devices in novel applications.

### SUMMARY OF INVENTION

Embodiments of the present invention are directed to solving these and other problems and disadvantages of the prior art, and provide systems and methods for measuring oxidation-reduction potential (ORP) characteristics (i.e., static oxidation- reduction potential (sORP) and/or the oxidation-reduction potential capacity (cORP)) of a fluid. Moreover, the measured ORP can provide information regarding the status of a subject rapidly and conveniently in a clinical and/or emergent setting.

Systems in accordance with embodiments of the present disclosure generally include a test strip with a reference cell, a sample chamber, and a plurality of electrodes. The sample chamber is configured to receive a fluid sample. The system additionally includes a readout device with contacts for interconnection to the electrodes of a test strip, a test signal power supply, and an electrometer. The readout device can additionally include memory and a processor operable to execute programming code stored in the memory to operate the power supply and record a voltage sensed by the electrometer over time.

More particularly, a system for identifying an oxidative capacity of a fluid in accordance with embodiments of the present disclosure can include:

a fluid sample;

a test strip, including:
a reference cell;
a sample chamber;
a counter electrode, wherein a first portion of the counter electrode extends into the sample chamber;
a working electrode, wherein a first portion of the working electrode extends into the sample chamber;
a reference electrode, wherein the reference electrode is in electrical contact with the reference cell;

a readout device, including:
a first contact;
a second contact;
a third contact;
a test signal power supply, wherein a first terminal of the test signal power supply is electrically connected to the first contact, and wherein a second terminal of the test signal power supply is electrically connected to the second contact;
an electrometer, wherein a first input of the electrometer is electrically connected to the second contact, and wherein a second input of the electrometer is electrically connected to the third contact;
memory;
a processor, wherein with the first contact electrically connected to the counter electrode, the second contact electrically connected to the working electrode, and the third contact electrically connected to the reference electrode, and with the fluid sample in the sample chamber, the processor is operable to execute programming code stored in the memory to:

operate the test signal power supply to supply a current across the sample chamber between the counter electrode and the working electrode for at least a first period of time;

during the first period of time, monitor the voltage sensed by the electrometer;

identify an inflection point in the voltage sensed by the electrometer;

record the time at which the inflection point is identified.

The processor can also operate to integrate the current between a start time and the time at which the inflection point is reached to obtain an oxidation-reduction capacity of the fluid sample. Alternatively or in addition, the processor can operate the test signal power supply to supply the current at a static first level for a first time segment, and after the first time segment, operate the test signal power supply to supply the current at a rising level for at least a second time segment.

The system can additionally include a readout device incorporating an output device, wherein the obtained oxidation-reduction capacity of the fluid sample is output to a user by the output device. The output can be provided in units of Coulombs⁻¹.

Methods in accordance with embodiments of the present disclosure include techniques for obtaining ORP characteristics of a fluid sample. The disclosed methods include applying a current to a fluid sample, and measuring a voltage across that fluid sample over a period of time while the current is applied. An inflection or transition point, such as a point at which the voltage is changing the fastest, can be identified. The quantity of current applied between the first period of time and the inflection point can then be integrated to obtain a value with units of Coulombs that is indicative of an oxidation- reduction capacity of the fluid sample. The determined value can then be output, for example for diagnostic or other purposes.

In accordance with embodiments of the present disclosure, a method for measuring oxidation-reduction potential capacity is disclosed that includes:

applying a current to a fluid sample;

measuring a voltage across the fluid sample over a first period of time, while applying the current to the fluid sample;

integrating the applied current over the first period of time to obtain a value indicative of an oxidation reduction capacity.

The current can be applied to the fluid sample between a counter electrode and a working electrode, and the voltage across the fluid sample can be measured between a reference electrode and the working electrode.

In accordance with at least some embodiments of the method, the current applied to the fluid sample is varied over time.

The inflection point in the measured voltage can be identified. In addition, the first period of time over which the current is integrated can end at a time at which the inflection point is identified.

The current can be held constant during at least a first segment of the first period of time, and the current can be varied during at least a second segment of the first period of time. The first segment of the first period of time can follow the second segment of the first period of time. Moreover, the current can be increased at a linear rate during the second segment of the first period of time. Alternatively, the current can be increased at an exponential rate during the second segment of the first period of time. As yet another alternative, the current can be increased according to a step function during the second segment of the first period of time.

According to at least some embodiments, the inflection point is the point at which the rate of change in the measured voltage is at a local maximum.

In accordance with still other embodiments, a readout device is provided that includes a plurality of readout contacts, and an analog front end. The analog front end includes a current supply and an electrometer. The current supply is operable to provide a current to first and second contacts of the plurality of readout contacts. The electrometer is operable to read a voltage potential between the second contact and the third contact of the readout contacts.

The readout device can further include a controller and an analog to digital converter that connects the electrometer to the controller. In addition, the readout device can include a digital to analog converter that connects the controller to the current supply.

The controller can operate to determine an inflection point in the voltage potential, and to determine a quantity of charge supplied between a first time and the inflection point.

A user interface can be provided that includes a user output. The user output can provide an oxidation reduction potential capacity value that is derived from the determined quantity of the charge supplied between the first time and the inflection point.

A connector that is operative to receive a test strip containing a fluid sample can also be included in the readout device. The connector includes the readout contacts, and places the readout contacts in operative contact with leads of the test strip.

Another embodiment of the invention is a method for characterizing, diagnosing, evaluating or monitoring an individual that has suffered a stroke. The method includes measuring the sORP, cORP and/or icORP of a sample from the individual, comparing the one or more measured ORP values to comparable reference values, and based on the comparison, characterizing the individual with regard to the severity of the stroke, the likely survival outcome of the individual, and/or the individual's estimated length of stay in a medical facility. In one embodiment, the sample is obtained during initial contact of the individual with a medical professional, or upon admission to a medical facility. In other embodiments, a second ORP value is determined, or measured, from a second sample obtain from the individual at a time subsequent to obtainment of the first sample, and any change in the OPR value used to characterize the individual with regard to the severity of the stroke, the likelihood of survival or the individual's estimated length of stay in a medical facility.

In one embodiment, reference ORP values are from one or more individuals known to have survived a stroke. In one embodiment, the reference ORP values are from one or more individuals known to have suffered a mild stroke, a moderate stroke or a moderately severe stroke. In one embodiment, the reference ORP values are from one or more individuals known to have suffered a severe stroke.

In one embodiment, if the sORP value or icORP of the first sample is significantly lower than the one or more comparable reference values, or if the cORP value is significantly higher than the one or more comparable reference values, the individual is characterized as being unlikely to survive. In one embodiment, if the sORP value or icORP of the first sample is significantly greater than the one or more comparable reference values, or if the cORP value is significantly lower than the one or more comparable reference values, the individual is characterized as being likely to survive.

In one embodiment, if the sORP value or icORP of the first sample is significantly lower than the one or more comparable reference values, or if the cORP value is significantly higher than the one or more comparable reference values, the individual is characterized as having suffered a severe stroke. In one embodiment, if the sORP value or icORP of the first sample is significantly greater than the one or more comparable reference values, or if the cORP value is significantly lower than the one or more comparable reference values, the individual is characterized as not having suffered a severe stroke.

Another embodiment of the invention is a method of characterizing an individual who has suffered a stroke, comprising measuring the ORP value of a first sample obtained from the individual, measuring the ORP value of a second sample obtained from the individual subsequent to obtainment of the first sample, comparing the ORP values of the second sample with the ORP value of the first sample to determine if any change has occurred, and based on the change, if any, characterizing the individual with regard to the individual's likelihood of survival, the severity of the stroke, or the individual's estimated length of stay in a medical facility. In one embodiment, the change in ORP value, if any, is compared to one or more comparable reference values to characterize the individual. In one embodiment, the one or more reference values are the change in ORP value(s) in the first 24 hours post-stroke stroke in one or more individuals known to have survived a stroke. In one embodiment, the one or more reference values are the change in ORP value(s) in the first 24 hours post-stroke stroke in one or more individuals known to have suffered a mild stroke, a moderate stroke, or a moderately severe stroke. In one embodiment, the one or more reference values are the change in ORP value(s) in the first 24 hours post-stroke stroke in one or more individuals known to have suffered a severe stroke. In one embodiment, the time between obtainment of the first and second samples is at least 6 hours, at least 8 hours, at least 12 hours, at least 18 hours at least 24 hours or at least 30 hours.

In one embodiment, if the change in OPR, if any, between the sORP value, or icORP value, of the first sample and the sORP value, or icORP value, of the second sample is significantly greater than the one or more comparable reference values, or if the change, if any, between the cORP value of the first sample and the cORP value of the second sample is significantly less than the one or more comparable reference values, characterizing the individual as being unlikely to survive. In one embodiment, if the change, if any, between the sORP value, or icORP value, of the first sample and the sORP value, or icORP value, of the second sample is significantly greater than the one or more comparable reference values, or if the change, if any, between the cORP value of the first sample and the cORP value of the second sample is significantly less than the one or more comparable reference values, characterizing the individual as having suffered a severe stroke.

On embodiment of the invention is a method of estimating the length of stay in a medical facility for an individual who ha suffered a stroke, comprising measuring the sORP, cORP and or icORP of a sample from the individual, comparing the one or more measured ORP values to comparable reference values, and based on the comparison, estimating the individual's length of stay in a medical facility.

One embodiment of the invention is the use of the individual's characterization in developing a treatment plan for the individual. In one embodiment, the treatment plan includes pharmaceutical treatment. In one embodiment, the pharmaceutical treatment is fibrinolytic therapy.

One embodiment of the invention is the use of the individual's characterization in developing a care plan for the individual. In one embodiment, the care plan includes determining the type of medical facility best suited for treatment of the individual. In one embodiment, care plan includes determining when the individual may be transferred to another facility or may be discharged to home.

In all of the foregoing embodiments, the step of determining or measuring can be determining, or measuring, the sORP, the cORP or the icORP. In addition, the reference values in all of the foregoing embodiments can be one or more of a normal reference value, a condition specific reference value and a self reference value. Reference values can also be historic reference values obtained from previous individuals. They can also be reference values from a single individual or average, or mean, values calculated from a group of individuals.

Additional features and advantages of embodiments of the present disclosure will become more readily apparent from the following detailed description, particularly when taken together with the accompanying drawings.

### BRIEF DESCRIPTION OF THE DRAWINGS

FIG. 1 depicts components of a system for measuring the oxidation-reduction potential capacity of a fluid in accordance with embodiments of the present invention;

FIG. 2 illustrates components of a readout device in accordance with embodiments of the present disclosure;

FIG. 3 illustrates further aspects of a readout device in accordance with embodiments of the present disclosure;

FIG. 4 depicts a test strip in accordance with embodiments of the present invention;

FIG. 5 is a flowchart depicting aspects of a method for measuring oxidation- reduction potential capacity in accordance with embodiments of the present disclosure; and

FIG. 6 is a graph depicting a supplied current and a measured potential difference over time.

FIG. 7 is a graph comparing admission sORP values and type of stroke. The asterisk indicates p values<0.05 compared to Severe Stroke data, categories based on NIHSS scores.

FIG. 8 is a graph comparing admission cORP values and type of stroke. The asterisk indicates p values<0.05 compared to Severe Stroke data, categories based on NIHSS scores.

FIG. 9 is a graph showing a comparison of admission sORP with length of hospital stay for survivors. The solid diagonal line indicates the best Fit. The grey zone indicates 95% confidence limits. Dotted diagonal lines indicate 95% prediction limits. R-value=+0.303; p<0.05, significant Pearson's R correlation. R²-value=0.093. N=95

FIG. 10 is a graph showing a comparison of admission sORP with length of hospital stay for survivors. The solid diagonal line indicates the best Fit. The grey zone indicates 95% confidence limits. Dotted diagonal lines indicate 95% prediction limits. R-value=+0.305; p<0.05, significant Pearson's R correlation. R²-value=0.093. N=95

### DESCRIPTION OF EMBODIMENTS

Embodiments of the present invention provide systems and methods for measuring oxidation-reduction potential (ORP) characteristics (i.e., static oxidation-reduction potential (sORP) and/or oxidation-reduction capacity (cORP)) of a fluid that are suitable for rapid, routine clinical diagnostic testing and methods of using the system to evaluate or monitor the status of a subject. The system generally includes a test strip and a readout device. More particularly, embodiments of the present invention can determine the ORP characteristics of a body fluid of a patient in a convenient and timely manner. A biological sample of a patient that can be used in the method of invention can be any body fluid. Suitable body fluids include a blood sample (e.g., whole blood, serum or plasma), urine, saliva, cerebrospinal fluid, tears, semen, vaginal secretions, amniotic fluid and cord blood. Also, lavages, tissue homogenates and cell lysates can be utilized and, as used herein, “body fluid” includes such preparations. Preferably, the body fluid is blood, plasma, serum or cerebrospinal fluid. For head injuries, the body fluid is most preferably cerebrospinal fluid or plasma. In cases other than head injuries, the body fluid is most preferably plasma.

The test strip generally includes a substrate, a reference cell, a counter electrode, a working electrode, a reference electrode, and a sample chamber. In general, by placing a fluid sample in the sample chamber, an electrical connection is established between the reference cell, the counter electrode, the working electrode, and the reference electrode. The test strip can then be connected to a readout device, for the determination of a static ORP value and an ORP capacity value.

The readout device generally includes contacts to electrically interconnect the readout device to the various electrodes included in the test strip. In accordance with embodiments of the present disclosure, the readout device includes an analog front end. The analog front end generally functions to provide a controlled current that can be sent across the fluid in the sample chamber through an electrical connection to the counter electrode and the working electrode. In addition, the analog front end is operable to generate a voltage signal that represents the potential difference between the reference electrode and the working electrode. An analog to digital (ADC) converter is provided to convert the voltage signal representing the reference electrode to working electrode potential difference to a digital signal. A digital to analog converter (DAC) is provided to convert a digital control signal to analog signals in connection with the provision of the controlled current to the test strip. A controller interfaces with the ADC and the DAC. Moreover, the controller can include or comprise a processor that implements programming code controlling various functions of the readout device, including but not limited to controlling the current supply to the test strip, and processing the potential difference measurement signal. The controller can operate in association with memory. In addition, the readout device includes a user interface, and a power supply.

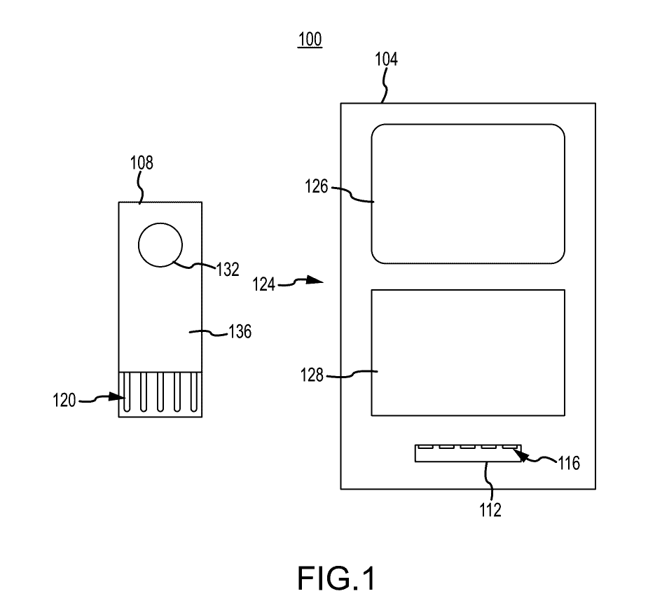

FIG. 1 depicts components of a system 100 for measuring the oxidation-reduction potential (ORP) value, including but not limited to the static oxidation-reduction value (sORP) and/or the oxidation-reduction capacity value (cORP), of a fluid sample in accordance with embodiments of the present disclosure. As used herein, the sORP is a measured potential difference or voltage across a fluid sample such as a measured potential difference or voltage across a fluid sample placed in a test strip that includes a reference cell as described herein. The cORP as used herein is a measure of the quantity of charge provided to a fluid sample over a defined period such as can be measured in a test strip as described herein. Accordingly, the cORP can be viewed as the capacity of a fluid sample to absorb an electrical charge supplied as a current over some defined period. For example, the period can be defined by a start point corresponding to the initiation of current supply to a sample and an endpoint such as an inflection point or a midpoint between a first and a second inflection point. In general, the system 100 includes a readout device 104, which can implement a galvanometer, and a test strip 108. The readout device 104 includes a connector or readout aperture 112 for electrically interconnecting readout contacts 116 of the readout device 104 to electrode contacts 120 provided as part of the test strip 108. The readout device 104 can also incorporate a user interface 124, which can include a user output 126, such as a display, and a user input 128, such as a keypad. In accordance with still other embodiments, the user interface 124 can comprise an integrated component, such as a touch screen interface. In addition to providing contacts 120 for interconnecting the test strip 108 to the readout device 104, the test strip 108 includes a sample chamber aperture 132 formed in a test strip overlay 136, to receive a fluid sample in connection with the determination of an ORP value of that fluid sample.

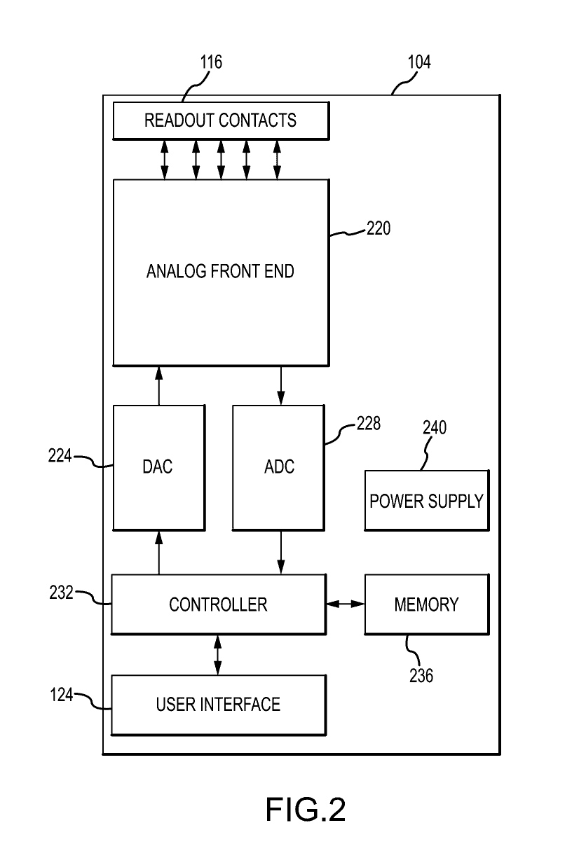

FIG. 2 illustrates additional components and features of a readout device 104 in accordance with embodiments of the present disclosure. As shown, the readout contacts 116 are interconnected to an analog front end 220. As described in greater detail elsewhere herein, the analog front end 220 generally functions to provide a controlled current that is passed between a counter electrode and a working electrode of the test strip 108. In addition, the analog front end 220 functions to provide a voltage signal representing a potential difference between a reference electrode and the working electrode of the test strip 108. In accordance with still further embodiments, the analog front end 220 can include a strip detect circuit, to provide a signal indicating the interconnection of a test strip 108 to the readout device 104.

The analog front end 220 generally receives control signals from a digital to analog (DAC) converter 224. Signals output by the analog front end 220 are generally provided to an analog to digital converter (ADC) 228. The DAC 224 and ADC 228 are in turn connected to a controller 232. The controller 232 may comprise a processor that is operable to execute instructions stored in memory as part of the controller 232, or as a separate memory device 236. For example, the processor, executing instructions stored in memory 236, can implement a process according to which the current supplied to the test strip 108 is controlled. In addition, the controller 232 can execute instructions stored in memory 236 to record the quantity of current supplied to the test strip 108, to detect an inflection point in the voltage potential between electrodes of the test strip 108, and to calculate an ORP capacity. The memory 236 can also function as storage for data, including but not limited to intermediate and/or final ORP values. The controller 232, for example, can comprise a general purpose programmable processor or controller or a specially configured application integrated circuit (ASIC).

The user interface 124 generally operates to provide user input to the controller 232. In addition, the user interface 124 can operate to display information to a user, including but not limited to the status of the readout device 104 or of the system 100 generally, a sORP value, and a cORP value.

The readout device 104 also generally includes a power supply 240. Although not shown in the figure, the power supply 240 is generally interconnected to power consuming devices via a power supply bus. The power supply 240 may be associated with a battery or other energy storage device, and/or line power.

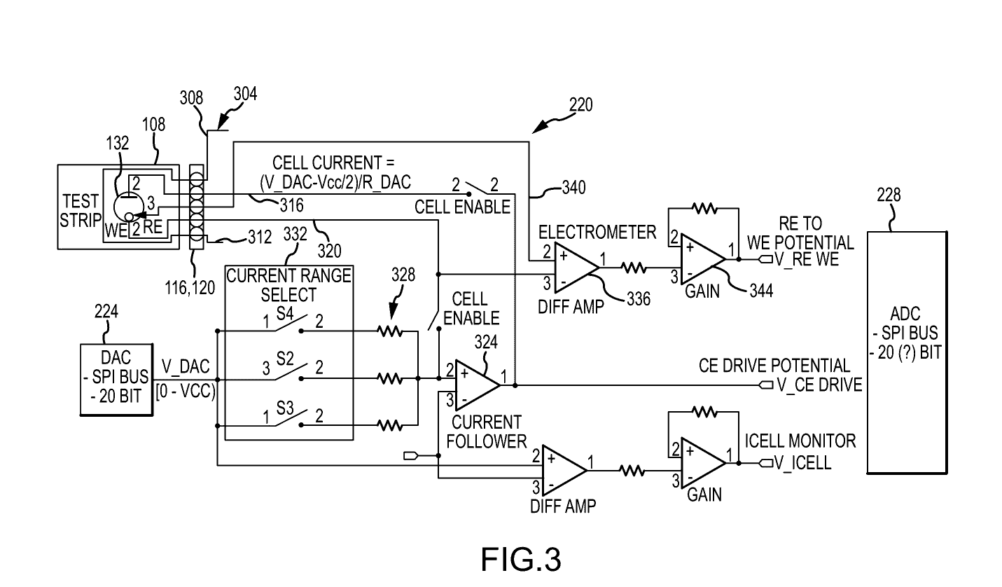

With reference now to FIG. 3, additional features of a system 100 in accordance with embodiments of the present disclosure are depicted. More particularly, details of the analog front end 220 and of the electrical circuit associated with the test strip 108 are depicted. As shown, the readout contacts 116 interconnect to the electrode leads or contacts 120, to electrically connect the analog front end 220 to the test strip 108. In the illustrated embodiment, the analog front end 220 includes a test strip sense circuit 304. The test strip sense circuit 304 includes a test strip detection supply lead 308 and a test strip detection input lead 312. In general, when a suitable test strip 108 is operatively connected to the readout device 104, continuity between the test strip detect supply lead 308 and the test strip detection input lead 312 is established, allowing a test strip detect signal indicating that a test strip 108 is present to be passed between the supply 308 and the input 312 leads. Moreover, a test strip 108 can incorporate a resistor or other component to modify the test strip detect signal, to indicate to the readout device 104 characteristics of the particular test strip 108 that has been interconnected to the readout device 104, such as the voltage value of a reference cell incorporated into the test strip 108. In response to sensing the presence of a test strip 108, the readout device 104 can operate to provide an interrogation signal in the form of a controlled current to the test strip 108.

The current is provided by the readout device 104 to the sample chamber 132 of the test strip 108 via a counter electrode lead 316 and a working electrode lead 320. More particularly, the current may be supplied to the counter electrode lead 316 from the output of a current follower 324, while the working electrode 320 can be provided as an input to that current follower 324. In addition, a set of current range select resistors 328 and associated switches 332 can be controlled by the DAC 224, as directed by the controller 232, for example depending on the characteristics of the interconnected test strip 108. In addition, the DAC 224, as directed by the controller 232, can control the input to the current follower 324 to in turn control the amount of current supplied to the test strip 108 by the current electrode lead 316. The DAC 224, as directed by the controller 232, can also operate various switches and/or amplifiers to control the operating mode of the analog front end 220.

The analog front end 220 additionally includes an electrometer 336 that receives a first input signal from a reference electrode lead 340 and a second input signal from the working electrode lead 320. The output from the electrometer 336 generally represents the potential difference between the reference electrode lead 340 and the working electrode lead 320. The signal output by the electrometer 336 can be amplified in a gain circuit 344, and output to the ADC 228.

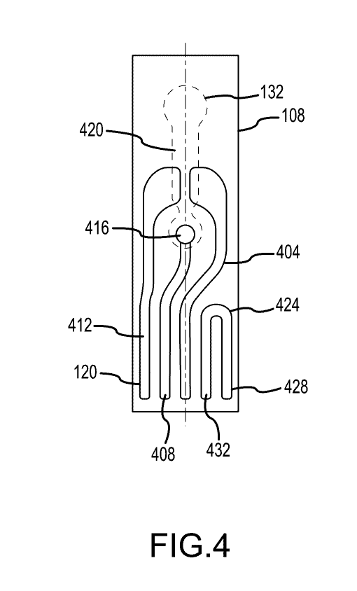

FIG. 4 depicts aspects of a test strip 108 in accordance with embodiments of the present invention. More particularly, the view presented by FIG. 4 shows the test strip 108 with the test strip overlay 136 removed. In general, the test strip 108 includes a working electrode 404, a reference electrode 408, and a counter electrode 412. In addition, the test strip 108 includes a reference cell 416. By placing a fluid sample within a sample chamber region 420, the working electrode 404, the reference electrode 408, the counter electrode 412, and the reference cell 416 are placed in electrical contact with one another. Moreover, by placing the electrode contacts 120 corresponding to the counter electrode 412, the working electrode 404 and the reference electrode 408 in contact with the readout contacts 116 corresponding to the counter electrode lead 316, the working electrode lead 320, and the reference electrode lead 340 respectively, the test strip 108 is operatively connected to the readout device 104. Accordingly, a supply current provided to the test strip 104 can be sent across the fluid sample, between the counter electrode 412 and the working electrode 404 by the readout device 104. Moreover, the potential difference between the reference electrode 408 and the working electrode 404 can be sensed by the readout device 104. In accordance with further embodiments of the present disclosure, the test strip 108 can include a test strip detect circuit 424, that includes an input 428 and an output 432. The test strip detect circuit 424 can, in addition to the input 428 and the output 432, include a resistor or other component for modifying a test strip sense signal provided by the readout device 104, to indicate to the readout device 104 an identification of the test strip 108.

To measure the cORP or antioxidant reserve, the sample is titrated with a linearly increasing oxidizing current between a counter and working electrode to exhaust the relevant antioxidants at the working electrode while monitoring the voltage between the working and reference electrodes. The result is a time vs voltage curve and a time vs 5753-28 current curve. The time versus voltage curve is used to find an inflection point where the voltage is changing the fastest (antioxidants are exhausted so system tries to find a new equilibrium). The time at maximum velocity (i.e., at the inflection point) is referred to as the transition time. The capacity or cORP is then the integral of the current profile from the beginning to the transition time with units of uC.

Calculation of the transition time may be accomplished several ways including noise filtration, curve fitting and standard numerical differentiation techniques. Usually the unfiltered numerical derivative is noisy, making finding maxima difficult or unreliable. To that end one technique is to curve fit the time versus voltage profile with a polynomial (5th-7th order is usually sufficient) and directly differentiating the resulting polynomial analytically. This approach has the advantage of very smooth derivatives making the determination of the transition time robust as long as the fit is good.

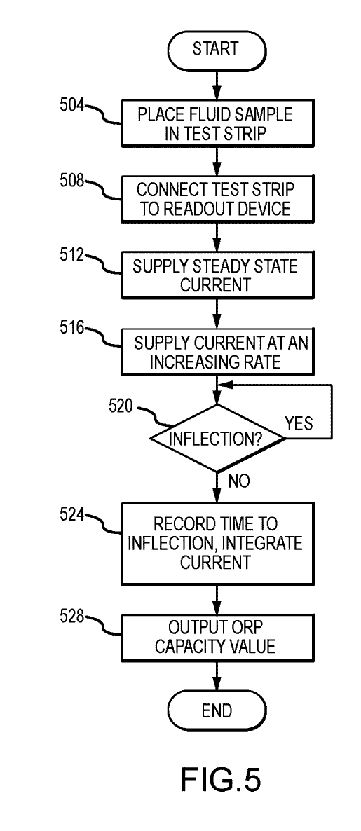

FIG. 5 is a flowchart illustrating aspects of the operation of a system 100 for determining the ORP, including but not limited to the cORP, of a fluid sample in accordance with embodiments of the present invention. In general, the method includes obtaining a fluid sample and placing the fluid sample in the sample chamber 420 of a test strip 108 (step 504). At step 508, the test strip 108 is connected to the readout device 104 (step 508). In general, while the readout device 104 is in an on or standby mode, an electrical signal may be output by the test strip detection output lead 308. By connecting a suitable test strip 108 to a readout device 104, continuity between the test strip detect output lead 308 and the test strip detect input lead 312 is established. In addition, the signal received at the test strip detect input lead 312 can provide an indication of characteristics of the test strip 108, which can in turn be used to control aspects (e.g., a current range) of a current supplied to the test strip 108. Such characteristics can include but are not limited to the type and composition of the test strip electrodes 404, 408 and 412, and the potential of the reference cell 416.

At step 512, a current can be supplied by the readout device 104 to the counter electrode 412 of the test strip 108. More particularly, a current can be passed between the counter electrode 412 and the working electrode 404 by the counter electrode lead 316 and the working electrode lead 320. In accordance with embodiments of the present disclosure, the current that is supplied to the test strip 108 is controlled by the controller 232 of the readout device 104. More particularly, the current can be provided for at least a first segment of time at a selected, steady state level. The first segment of time can be a fixed time period. Alternatively, the first segment of time can expire once a determination has been made that the potential difference sensed by the readout device 104 between the reference electrode 408 and the working electrode 404 has a rate of change that is less than some selected amount. In accordance with still other embodiments, a combination of parameters may be applied to determine the time period over which the current is supplied at a steady state. Moreover, in accordance with other embodiments, no current is supplied during the first period of time (i.e. the supplied current during the first segment of time is zero). As can be appreciated by one of skill in the art after consideration of the present disclosure, while no current is supplied and while the rate of change of that potential difference is zero or less than some selected amount, the potential difference measured by the readout device 104 between the reference electrode 408 and the working electrode 404 is equal to the sORP of the fluid sample.

After the first segment of time has expired, the current can be supplied at an increasing rate (step 516). For example, the amount can be increased linearly, as a step function, exponentially, according to a combination of different profiles, or in any other fashion. For instance, the current can be increased linearly from 0 amps at a specified rate until an endpoint is reached. As another example, the amount can be stepped from 0 amps to some non-zero value, and that non-zero value can be provided at a steady rate for some period of time, or can be provided at an increasing rate according to some function. At step 520, a determination can be made as to whether an inflection point in the potential difference monitored between the reference electrode 408 and the working electrode 404 has been detected. More particularly, the reference electrode lead 340 and the working electrode lead 320 connect the reference electrode 408 and the working electrode 404 respectively to the electrometer 336, which outputs a signal representing the potential difference between the reference 408 and the working 404 electrodes. The analog to digital converter 228 then converts the signal representing the potential difference between the reference 408 and working 404 electrodes to a digital signal that is provided to the controller 232. If an inflection point has been detected, the readout device 104, and in particular the controller 232, can record the time from which current was first supplied to the time at which the inflection point is reached. In addition, the controller 232 can integrate the current signal to determine an amount of charge that has been supplied to the fluid sample up to the time at which the inflection point is reached (step 524). In accordance with embodiments of the present disclosure, a first inflection point (e.g., a point at which the voltage measured across a fluid sample while a current is being supplied is at a local maximum rate of change) is used as the point at which integration of the current is stopped. However, multiple inflection points can be observed in the measured voltage. Accordingly, rather than using the first observed inflection point as the end point for integration, a subsequent inflection point can be used. As yet another example, a time determined with reference to multiple inflection points, such as a midpoint between two observed inflection points or an average time of multiple observed inflection points can be used as the end point of the integration for purposes of determining the cORP of a fluid sample. At step 528, the determined quantity of charge or a value derived from the determined quantity of charge can be output to a user as an ORP capacity (cORP) value for the fluid sample, for example through the output device 128 facility of a user interface 124 provided as part of or interconnected to a readout device 104. For example, the cORP value can be defined as one over the quantity of charge. The process can then end.

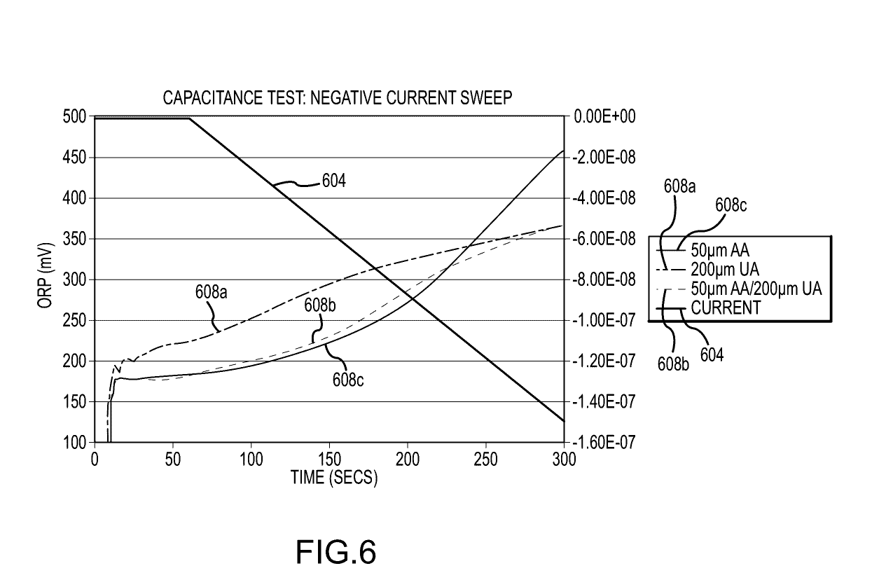

FIG. 6 depicts the current, shown as line 604, supplied by a readout device 104 to an interconnected test strip 108 over time. In addition, sample measured potential difference values 608a-c for different exemplary samples are depicted. As can be understood by one of skill in the art after consideration of the present disclosure, although three potential difference values 608 are shown, a current 604 is provided to only one fluid sample during determination of an ORP value. As can also be appreciated by one of skill in the art after consideration of the present disclosure, the ramped portion of the current 604 is shown sloping in a downward direction, because it depicts an oxidizing current. In addition, it can be appreciated that the area between the current curve 604 and a current value of zero for a selected period of time represents a quantity of charge provided to a fluid sample held in a test strip 108. Accordingly, this quantity of charge can be used to provide a measurement of the ORP capacity (cORP) of the fluid sample. Moreover, the voltage curves 608 represent a static ORP (sORP) value of a respective fluid sample at different points in time. The area under the current curve 604 (which is above the curve 604, between that curve and a current of zero in FIG. 6) that is used to determine the cORP can have a start point at a first point in time and an end point at a second point in time. As an example, the start point for integration of the current 604 can be selected as a point at which the observed sORP signal or reading has stabilized. For instance, in the example of FIG. 6, the potential difference values have stabilized after about 50 seconds have elapsed. Moreover, in this example no current is being supplied to the sample by the readout device 104 during the first segment of time leading up to the start point at which current is supplied. That start point can also correspond to the time at which the current 604 begins to be applied at an increasing rate. In accordance with embodiments of the present disclosure, where a curve 608 reaches an inflection point, for example the point at which the rate of change in the measured potential difference is at a maximum (i.e., a point of maximum slope), the integration of the current signal 604 is stopped. For example, looking at curve 608b, an inflection point can be seen at about 200 seconds, and integration of the current 604 can thus be performed during the period beginning at 50 seconds and ending at 200 seconds. Alternatively, the integration of the current signal 604 can be stopped after some predetermined period of time. As yet another alternative, the integration of the current signal 604 can be stopped at the earlier of the observation of an inflection point or the expiration of a predetermined period of time.

As can be appreciated by one of skill in the art after consideration of the present disclosure, the measurement of the sORP value can be in units of Volts, and the integration of the current signal or value 604 therefore gives a value representing a quantity of charge in Coulombs. cORP values, as a measure of a quantity of charge, is expressed herein as one over the quantity of charge in Coulombs. In particular, by taking the inverse of the observed quantity of charge, a more normal distribution is obtained, facilitating the application of parametric statistics to observed ORP values. As used herein, the terms ORP capacity, inverse capacity levels, inverse capacity ORP or ICL are all equivalent to cORP as defined above. It will be appreciated that expression of cORP as one over a quantity of charge encompasses alternative equivalent expressions.

As noted above, higher than normal values of sORP are indicative of oxidative stress and are considered to be a negative indication for the subject being evaluated. cORP is a measure of a subject's capacity to withstand oxidative insult. Thus, it is a positive indication for a subject to have a normal or higher capacity to withstand oxidative insult. Since cORP is defined as the inverse of the quantity of charge to reach a voltage inflection point, a higher cORP value is indicative of a lesser capacity to withstand oxidative insult, and likewise, a lower cORP value is indicative of a greater capacity to withstand oxidative insult.

The present invention includes embodiments for monitoring or evaluating the health of patients having a variety of conditions by determining the ORP characteristics of a biological sample of the patient. Typically, the ORP characteristics of the patient are compared to an ORP characteristic reference value or values that are relevant to that patient. As used herein, a reference value can be an ORP characteristic of the patient from a time when the patient did not have the condition in question (i.e., when he/she was healthy) or from an earlier time period when the patient had the condition in question (for purposes of monitoring or evaluating the condition or treatment thereof). Such reference values are referred to as self reference values. For example, reference values can also include initial, maximum and ending reference values, such as when ORP characteristics are evaluated over a time frame such as when a patient is being admitted to a medical facility (initial), during a stay at a medical facility (maximum), and at a time when a patient is being considered for transfer, discharge, or other disposition (ending). Alternatively, a reference value can be an ORP characteristic of a relevant healthy population (e.g., a population that is matched in one or more characteristics of species, age, sex, ethnicity, etc.). Such reference values are referred to as normal reference values. Further, a reference value can be an ORP characteristic of a relevant population similarly situated as the patient (e.g., a population having the same or similar condition as the patient for which the patient is being treated and preferably, one that is also matched in one or more characteristics of species, age, sex, ethnicity, etc.). Such a reference value is referred to as a condition specific reference value. For example, a condition specific reference value can be a reference value from one or more stroke patients. Further, such reference values can be correlated with information about the patients from which they were obtained. For example, a reference value from stroke patients can be correlated with the type of stroke suffered by the patient, the patients' outcome, and/or the patients' length of stay in a medical facility.

As used herein, an individual, subject, patient, and the like, is any individual for whom a biological sample is being tested for an ORP characteristic. The term subject can include a patient if the subject is an individual being treated by a medical professional. The terms subject and patient can refer to any animal, including humans and non-human animals, such as companion animals (e.g., cats, dogs, horses, etc.) and livestock animals (i.e., animals kept for food purposes such as cows, goats, chickens, etc.). Preferred subjects include mammals and most preferably include humans.

In various embodiments of the invention, the ORP characteristics of a biological sample of a subject are measured. The measurement of the ORP characteristics of a biological sample can be done at multiple time points. The frequency of such measurements will depend on the condition being evaluated. For example, urgent conditions such as stroke can employ more frequent testing of an individual. In contrast, chronic conditions such as neurodegenerative conditions can employ longer term testing intervals. As such, for example, testing can be done every 30 minutes, hour, 2 hours, 3 hours, 4 hours, 5 hours, 6 hours, 12 hours, 18 hours, or every day, for more urgent conditions. Alternatively, testing can be done every day, 2 days, 3 days, 4 days, 5 days, 6 days, week, 2 weeks, 3 weeks, month, 2 months, 3 months, 4 months, 5 months, 6 months, 7 months, 8 months, 9 months, 10 months, 11 months, or year for more chronic conditions.

In various embodiments of the invention, the ORP characteristics of a biological sample of a subject are measured for purposes of characterizing, diagnosing, evaluating or monitoring a subject for a specific condition, such as stroke. In such embodiments, the methods can include identifying in the subject a risk factor, such as a lifestyle or genetic risk factor, for the specific condition and/or a symptom of the specific condition.

As used herein, a medical facility is any facility at which an individual who has suffered a stroke can obtain care for the stroke from a medical professional. Examples of such medical facilities include, but are not limited to, hospitals, emergency rooms, urgent care facilities, outpatient facilities, nursing homes, residential treatment centers, skilled nursing facilities, and geriatric care facilities.

A medical professional is any individual having some level of training in delivering medical care. Such an individual is likely, but need not be, associated with a medical facility. Examples of such medical professionals include, but are not limited to, physicians, physician assistants, nurse practitioners, nurses, paramedics, emergency medical technicians, and skilled medical technicians and assistants.

One embodiment of the present invention is a method of characterizing, diagnosing, evaluating or monitoring an individual who has suffered a stroke, wherein the characterization, diagnosis, evaluation or monitoring is based on the ORP status of the individual. A general method of the invention can be practiced by measuring the ORP value of one or more biological samples from an individual who has suffered a stroke, and then evaluating if the ORP value is significantly different than one or more reference values. Preferred reference values are ORP values determined from samples obtained from one or more individuals known to have suffered a stroke, whose outcome is known and/or whose length of stay in a medical facility can be determined. In addition, it may be determined if the ORP value has increased or decreased compared to a prior ORP value obtained from the same individual. Any increase or decrease may also be compared to appropriate reference values. The subject can then be characterized, diagnosed, evaluated or monitored based on the information obtained from such comparisons.

One embodiment of the present invention is a method of characterizing an individual who has suffered a stroke, the method comprising:
a. measuring the ORP value of a biological sample obtained from the individual; and,
b. using the measured ORP value to characterize the individual.
   In one embodiment, characterization of the individual comprises determining the individual's likelihood of survival. In one embodiment, characterization of the individual comprises determining if the individual suffered a severe stroke. In one embodiment, characterization of the individual comprises estimating the individual's length of stay in a medical facility.

According to the present invention, the ORP value of the subject can be obtained from a biological sample of the subject, including but not limited to blood, plasma, serum, and cerebrospinal fluid (CSF) in a convenient and timely manner. The ORP value can also be obtained from a tissue of the subject, including but not limited to, brain tissue biopsy.

Without intending to the limit the definition, a stroke is generally defined as interruption of blood flow to one or more parts of the brain (i.e., ischemia). Without proper blood flow to supply oxygen and nutrients, and to remove waste products, brain cells in the ischemic region begin to die. Depending on the region of the brain affected, a stroke may cause, for example, paralysis, speech impairment, loss of memory and reasoning ability, coma, or death. A stroke can also be referred to as a brain attack or a cerebrovascular accident (CVA). As used herein, a stroke patient is a patient in the care of a medical system who is diagnosed, or is suspected of having, a stroke.

As described above, methods of the present invention involve, at least, measuring the ORP value of a biological sample taken from a stroke patient upon admission to a medical system. According to the present invention, terms such as, for example, admission to a medical system, in the care of a medical system, and the like, refer to the delivery of care by a medical professional. A medical professional is a person who has received training in the delivery of medical care. As such, medical professionals include, but are not limited to, emergency medical technicians (EMTs), paramedics, medics, nurses, nurse practitioners, physician assistants, and the like. Accordingly, admission to a medical system need can, but need not, occur in a structured facility, such as a hospital. For example, initiation of care by a paramedic at home or accident scene would be considered admission into medical care or a medical system. Thus, as used herein, an admission ORP refers to an ORP value in a sample obtained upon admission to medical care or admission to a medical system. In this regard, it should be noted that admission ORP can also be referred to as initial ORP (from an initial sample). In one embodiment, the initial biological sample, and/or ORP, is obtained away from a hospital. In one embodiment, the initial biological sample and/or ORP, is obtained in a hospital or emergency facility.

As has been described herein, ORP can refer to static ORP (sOTRP) or capacity ORP (cORP). In one embodiment, the ORP being measured is sORP. In one embodiment, the ORP being measured is cORP. It should also be appreciated that the ORP value can be reported as inverse capacity ORP (icORP), which is the inverse of cORP. Thus, it should be understood methods of the present invention can also be practiced using icORP. In one embodiment, the sORP, the cORP and/or the icORP is measured or determined.

One embodiment of the present invention is a method of determining the likelihood of survival of a stroke patient, comprising:
a. measuring the ORP value of a biological sample obtained from an individual who has suffered a stroke; and,
b. using the measured OPR value to determining the stroke patient's likelihood of survival.

In some embodiments, methods of determining the likelihood of survival of a stroke patient are based on the ORP of a biological sample obtained during initial contact of the individual with a medical professional. For example, the medical professional can be an EMT or paramedic. In another example, the medical professional is a nurse, doctor, or hospital technician. In one embodiment, the ORP is an admission ORP. Such methods are based on data provided by the inventor demonstrating a relationship between the admission ORP value, the severity of the stroke and the likelihood of survival of the stroke patient. In particular, the inventors have shown that, in general, the initial ORP value (e.g., admission ORP value) is proportional to the severity of the stroke. However, the inventors have also shown that in cases of severe stroke, the initial ORP value (e.g., admission ORP value) is less than the ORP value observed in patient's having a mild, moderate or moderately severe stroke. The inventors have also shown that patient's having admission ORP values less than the ORP value observed in patient's having a mild, moderate or moderately severe stroke, are less likely to survive the stroke. Thus, in one embodiment, the stroke patients' likelihood of surviving the stroke is determined by comparing the stroke patient's admission ORP value to a comparable reference value. According to the present disclosure, a comparable reference value is an ORP value obtained from a similar type of sample. (e.g., blood sample, urine sample, CSF sample, etc.) Further, a comparable reference value is an ORP value from a sample obtained at approximately the same time as the sample being measured (e.g., admission sample, 6 hour sample, 12 hour sample, 24 hour sample, etc.) Finally, a comparable reference value should be the same type of ORP value as the measured OPR value (e.g., sORP, cORP or icORP). In an embodiment in which the patient is being characterized with regard to likelihood of survival, a suitable reference value is one or more reference values from comparable samples obtained from one or more individuals known to have survived a stroke. In one embodiment, the reference value is one or more ORP values from comparable samples obtained from one or more stroke patients selected from the group consisting of patients known to have suffered a mild stroke, patients known to have suffered a moderate stroke, and patients known to have suffered a moderately severe stroke.

A low admission value indicates the stroke patient is unlikely to survive. Thus, in one embodiment, if the measured sORP value of the sample is significantly lower than one or more comparable reference values, the individual is characterized as being unlikely to survive. In one embodiment, if the measured icORP value of the sample is significantly lower than one or more comparable reference values, the individual is characterized as being unlikely to survive. In one embodiment, if the measured cORP value of the sample is significantly higher than one or more comparable reference values, the individual is characterized as being unlikely to survive. As used herein, and particularly with regard to comparison of ORP values, the term significantly refers to a difference of at least at least 10% or at least at least 15%. In one embodiment, an admission ORP value of about 130 mV, or less, indicates the stroke patient is unlikely to survive. In one embodiment, an admission ORP value of about 145 mV, or more, indicates the stroke patient is unlikely to survive. As used herein, the word about refers to a variation in ORP value of less than 15%, less than 10%, or less than 5%.

The inventors have also discovered that changes in ORP values can be used to determine a stroke patients' outcome with regard to survival. In particular, the inventors have discovered that the ORP value of a sample taken from a patient during the 24 hours following the stroke event can be used to determine the patient's outcome. Thus, in one embodiment of the invention, the ORP value of a second biological sample taken subsequent to the first biological sample is determined, and compared to the ORP value of the first biological sample to determine if there is a change in ORP value. In preferred embodiments, the time between obtainment of the first and second samples is at least 6 hours, 12 hours, 18 hours, 24 hours or 30 hours. The likelihood of survival of the individual suffering the stroke is then determined based on any change observed. In this regard, the inventors have discovered that individuals having the largest change in ORP in the 24 hours following the stroke are less likely to survive. Conversely, individuals having the smallest change in ORP over the first 24 hours are more likely to survive. To determine the likelihood of survival, changes in ORP can be compared comparable reference values.

In one embodiment, the individual's likelihood of surviving the stroke is determined by comparing a change in an individual's ORP value, if any, to one or more comparable reference values. In one embodiment, the one or more reference values are obtained from comparable samples obtained from one or more stroke patients selected from the group consisting of patients known to have suffered a mild stroke, patients known to have suffered a moderate stroke and patients known to have suffered a moderately severe stroke. In one embodiment, the one or more reference values are obtained from comparable samples obtained from one or more stroke patients who have suffered a severe stroke.

In one embodiment, a patient having a large change in ORP in samples obtained during the first 24 hours following a stroke is characterized as being unlikely to survive. In one embodiment, a patient having a small change in ORP in samples obtained during the first 24 hours following a stroke is characterized as being likely to survive.

In one embodiment, if a change in the measured sORP value of the sample is significantly greater than one or more comparable reference values, the individual is characterized as being unlikely to survive. In one embodiment, if the measured icORP value of the sample is significantly greater than one or more comparable reference values, the individual is characterized as being unlikely to survive. In one embodiment, if the measured cORP value of the sample is significantly less than one or more comparable reference values, the individual is characterized as being unlikely to survive.

In one embodiment, if a change in the measured sORP value of the sample is significantly less than one or more comparable reference values, the individual is characterized as being likely to survive. In one embodiment, if the measured icORP value of the sample is significantly less than than one or more comparable reference values, the individual is characterized as being likely to survive. In one embodiment, if the measured cORP value of the sample is significantly greater than one or more comparable reference values, the individual is characterized as being likely to survive.

In one embodiment, a patient having a change in ORP in samples obtained during the first 24 hours following a stroke of about 20 mV or more is characterized as being unlikely to survive. In one embodiment, a patient having a change in ORP in samples obtained over the first 24 hours following a stroke of less than about 17 mV is characterized as being likely to survive.

It should be appreciated that while admission ORP values and changes in ORP values of samples obtained during the first 24 hours following stroke can be used independently, they can also be used together. Thus, in one embodiment, a patient having a low initial, or admission, ORP and a large change in ORP over the first 24 hours following the stroke is characterized as being unlikely to survive. In one embodiment, a patient having an initial, or admission, ORP value of about 130 mV or less and a change in ORP over the first 24 hours following the stroke of about 20 mV, or more, is characterized as being unlikely to survive. In one embodiment, a patient having an admission ORP value of about 145 mV or more and a change in the ORP value of samples obtained during the first 24 hours following the stroke of about 17 mV, or less, is deemed as being likely to survive.

In one embodiment, the ORP characteristics measurement are taken in addition to other patient diagnostic criteria such as one or more of vital signs, ECG, blood sugar level, CT scan (CAT Scan, Computed axial tomography), MRI (Magnetic resonance imaging, MR), MRA (Magnetic resonance angiogram), Cerebral arteriogram (Cerebral angiogram, Digital subtraction angiography), PT (Prothrombin time) or PTT (Partial thromboplastin time), in order to diagnose stroke and/or rule out such diagnoses as drug overdose, hyper/hypoglycemia, seizure, head trauma, intracranial mass, migraine, meningitis, encephalitis, cardiac and arrest ischemia. Additionally, the ORP characteristics measurement may be used alone or in conjunction with the other diagnostic criteria described above to evaluate the use of fibrinolytic therapy or other acute interventions.

Methods of the present invention can also be used to predict the length of stay in a medical facility, such as a hospital, for individuals predicted to survive the stroke. Thus, one embodiment of the present invention is a method for identifying a stroke patient likely to have a longer hospital stay, the method comprising:
a) measuring the ORP of a sample obtained from an individual admitted to a medical facility for a stroke;
b) determining the individual's estimated length of stay by comparing the measured ORP value to one or more reference values.
   In one embodiment, the one or more reference values are obtained from one or more stroke patients selected from the group consisting of a patient admitted to a medical facility for a mild stroke, a patient admitted to a medical facility for a moderate stroke, and a patient admitted to a medical facility for a moderately severe stroke. In one embodiment, the individual is characterized as likely to have a longer hospital stay when the individual's measured ORP value is significantly higher than the appropriate reference value. In one embodiment, the individual is characterized as likely to have a longer hospital stay when the individual's measured ORP value is significantly higher than the ORP value obtained from a person admitted to a hospital for a mild stroke, the ORP value obtained from a person admitted to a hospital for a moderate stroke or the ORP value obtained from a person admitted to a hospital for a moderately severe stroke.

In one embodiment, the individual is characterized as likely to have a shorter hospital stay when the individual's measured ORP value is equal to or significantly lower than the appropriate reference value. In one embodiment, the individual is characterized as likely to have a shorter hospital stay when the individual's measured ORP value is significantly lower than the ORP value obtained from a person admitted to a hospital for a mild stroke, the ORP value obtained from a person admitted to a hospital for a moderate stroke or the ORP value obtained from a person admitted to a hospital for a moderately severe stroke. In one embodiment, the individual is characterized as likely to have a shorter hospital stay when the individual's measured ORP value is significantly lower than the ORP value obtained from a person admitted to a hospital for a severe stroke.

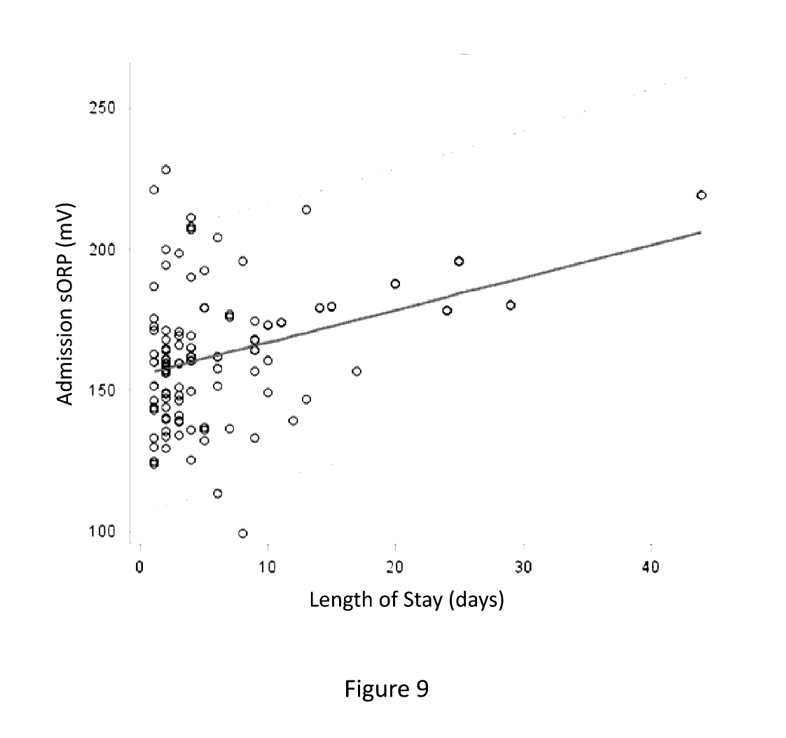

In one embodiment, the individual's length of stay in a medical facility is estimated using the data in FIGS. 9 and/or 10.

ORP characteristics of the subject that are statistically similar to, or greater than, the ORP characteristics of a subject or group of subjects diagnosed with stroke is indicative of a stroke in the subject and may indicate use of fibrinolytic therapy in the subject and/or admission to a hospital care unit. Alternatively, ORP characteristics of the subject that are statistically similar to a subject or group of “normal” subjects that are not affected by stroke is indicative of other sources of neurological distress and may suggest no intervention with fibrinolytic therapy in the subject.

An increase in the ORP characteristics of the subject over time following initial presentation of the subject may be indicative of developing brain damage in the stroke patient. In order to determine the trend of the ORP characteristics in the subject over time, without limitation, the ORP characteristics value of the subject may be checked every 1, 2, 3, 4, 5, 6, 7, 8, 9, or 10 minutes after the initial determination for a period of 1, 2, 3, or 4 hours, in order to compare and determine a trend in the ORP characteristics value of the subject. If the subject is admitted to a hospital care unit, the subject may be monitored at frequent intervals over the entire period of stay prior to discharge and in some embodiments, the change in the ORP characteristics values may be at least one factor considered in determining the appropriate discharge date.

In each of these embodiments, the ORP value from the subject is preferably obtained by applying a current to a biological sample (a fluid or tissue sample) obtained from the subject and measuring a voltage across the sample over a period of time. The measured voltage is integrated over the period of time to obtain a value indicative of an oxidation reduction capacity (ORP).

Accordingly, the present invention has been described with some degree of particularity directed to the exemplary embodiments of the present invention. It should be appreciated though that modifications or changes may be made to the exemplary embodiments of the present invention without departing from the inventive concepts contained herein.

### EXAMPLES

### Example 1

This Example demonstrates measurement of oxidation reduction potential (ORP) in patients admitted to a Primary Stroke Center with stroke symptoms, as a tool for assessing and correlating degrees of oxidative stress, severity of injury and relationship of ORP with outcomes.

Samples were obtained from patients admitted to the Primary Stroke Center between January of 2010 and December of 2012. Patients under the age of 18 were omitted, as were patients who had transferred into the facility. The static ORP (sORP) and capacity ORP (cORP) of each sample was measured using the RedoxSys diagnostic system, made by Luoxis (Englewood, Colo.). Patients were also clinically assessed using the National Institutes of Health Stroke Scale (NIHSS) and assigned a score based on the assessment. The demographics of the individuals included in the study are shown below in Table 1.

TABLE 1
Demographics of patients included in study

| | N (101 Total) | % |
|---|---|---|
| Variable | | |
| Age ≧55 | 83 | 82.2 |
| Female Gender | 59 | 58.4 |
| White Race | 79 | 78.2 |
| IV/IA Treatment | 24 | 23.8 |
| Surviving Stroke Patients | 87 | 93.1 |
| Hospital LOS (median, IQR) | 3.5 | 2-7 |
| Stroke Type | | |
| Hemorrhagic | 10 | 9.9 |
| Ischemic | 52 | 51.5 |
| TIA | 19 | 18.8 |
| Other | 20 | 19.8 |
| NIHSS | | |
| 0-4 (mild) | 38 | 50.0 |
| 5-15 (moderate) | 27 | 35.5 |
| 16-20 (moderately severe) | 7 | 9.2 |
| 21-42 (severe) | 4 | 5.3 |

The measured ORP values were compared with the type of stroke, demographics (e.g., age, gender, race, etc.), thombolytic therapy, NIH stroke scale and outcomes (e.g., in-hospital mortality, discharge modified Rankin Score, and length of stay (LOS)).

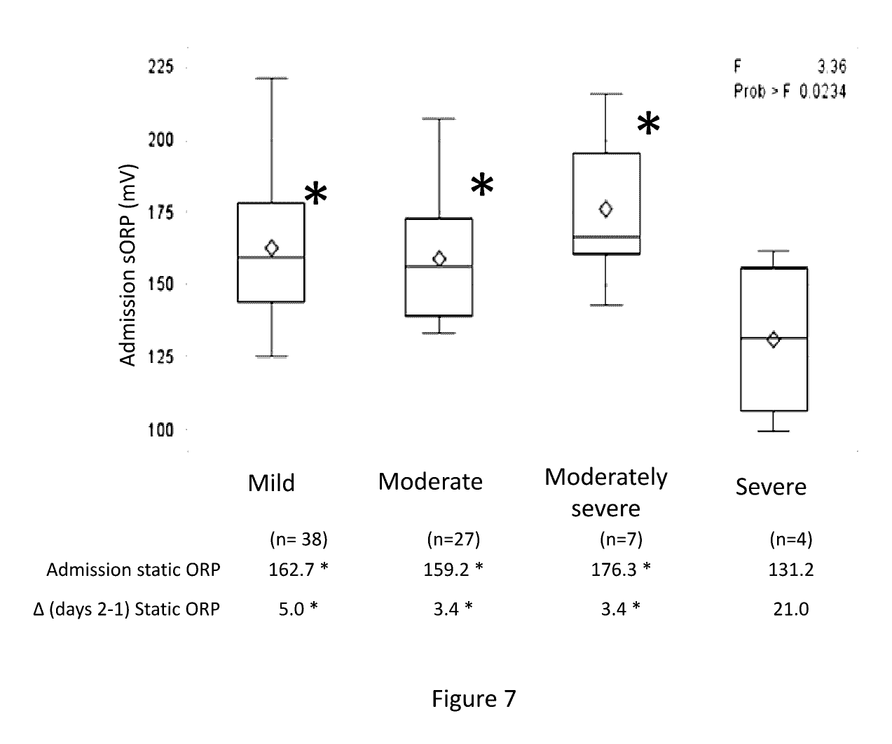

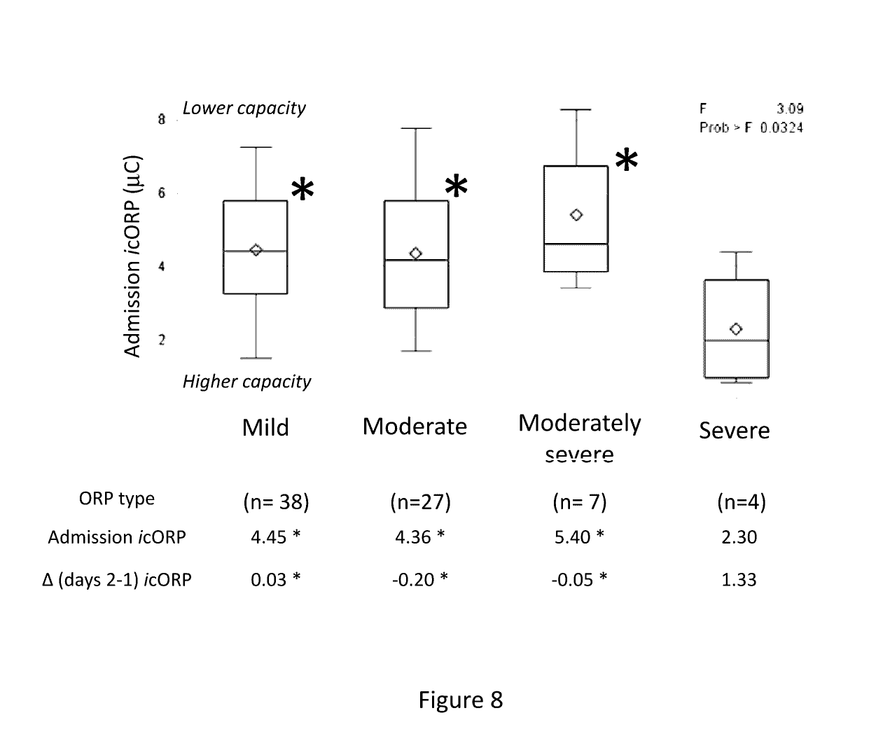

Initially, the patient's admission sORP and cORP values were compared with the type of stroke suffered by the patient. Stroke type was also compared with sORp and cORP values from samples taken 24 hours after admission. The results of these comparisons are shown in FIGS. 7 (sORP) and 8 (cORP). It should be noted that in FIG. 8, the cORP value is graphed as inverse cORP (icORP). The data demonstrate that the most sever stroke patients had the lowest sORP measurements and the highest ORP capacity (lower icORP) upon admission. It also shows that the most severe stroke patients had the largest 24 hour increase in sORP and the largest 24 hour decrease in cOPR (largest 24 increase in icORP).

Next, patient outcome (i.e., surviving or deceased) was compared with admission cORP and icORP and with changes in sORP and icORP in the 24 hours following admission. The results are shown below in Table 2.

TABLE 2
Comparison of patient outcome with admission
ORP and 24 hour change in ORP

| ORP Type | Surviving (n = 92) | Deceased (n = 5) |
|---|---|---|
| First sORP | 163.9 | 137.2* |
| Δ (Day 2 − day 1) | 5.37 | 26.34* |
| First icORP | 4.52 | 2.69* |
| Δ (Day 2 − day 1) | −0.01 | 1.49* |

*p values < 0.05 compared to surviving patients

The data demonstrate that surviving patients had higher sORP and icORP measurements at admission and smaller changes in ORP values in the 24 hours following admission.

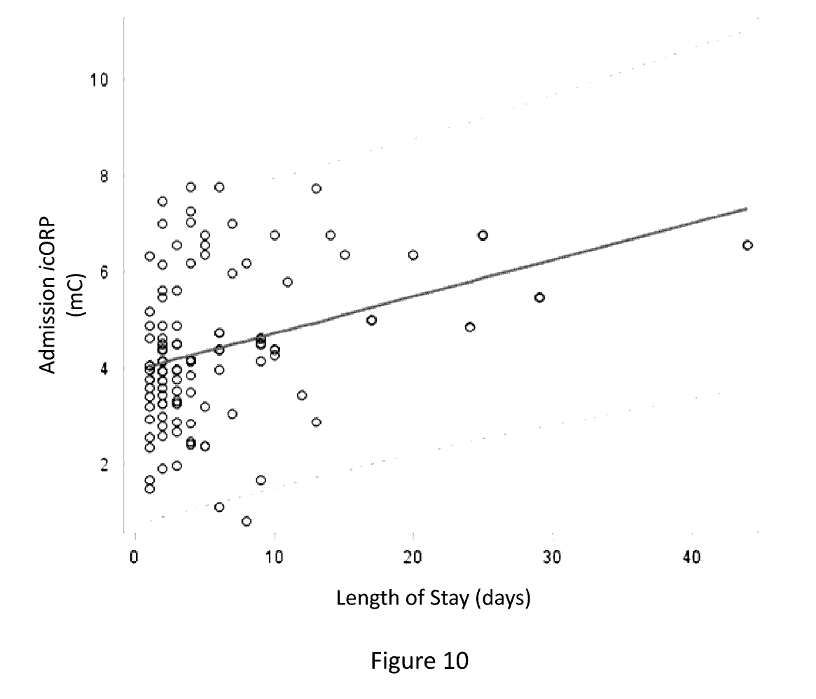

Finally, admission ORP values were compared with the lengths of stay in the hospital of surviving patients. These results of these comparisons are shown in FIGS. 9 and 10. FIG. 9 shows that higher levels of sORP at admission is associated with a longer hospital stay. Likewise, FIG. 10 shows that lower levels of capacity ORP (and therefore higher icORP) associates with longer hospital stays. In summary, the data from this study showed that at admission, severe stroke patients (NIHSS≧21) had a lower measure of oxidative stress (˜130 mV sORP) but that within 24 hours, they experienced large increases in the first 24 hours post admission (˜20 mV sORP). Their ORP values increased to levels similar to that of other stroke groups (mid, moderate, moderately severe). The data also shows that stroke survival is associated with higher measures of oxidative stress at admission, and in smaller changes in ORP values during the first 24 hours following a stroke. Moreover, stroke patients that eventually died had larger changes in OPR values during the first 24 hours. Thus, the data indicate that ORP measurements taken at admission and at 24 hours post-admission can be used to identify stoke patients at greatest risk and to estimate a patient's length of hospital stay.

The foregoing examples of the present invention have been presented for purposes of illustration and description. Furthermore, these examples are not intended to limit the invention to the form disclosed herein. Consequently, variations and modifications commensurate with the teachings of the description of the invention, and the skill or knowledge of the relevant art, are within the scope of the present invention. The specific embodiments described in the examples provided herein are intended to further explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed:

1. A method of determining the severity of stroke in an individual who has suffered a stroke, the method comprising:
a) measuring the ORP value of a first sample obtained from the individual, wherein the ORP value is a sORP value, a cORP value and/or a icORP value; and,
b) using the measured ORP value to determine if the individual suffered a severe stroke.

2. The method of claim 1, wherein the first sample is obtained during initial contact of the patient with a medical professional, or upon admission to a medical facility.

3. The method of claim 1, wherein step b) comprises comparing the measured ORP value of the first sample to a comparable reference ORP value to determine if the individual suffered a severe stroke.

4. The method of claim 3, wherein the reference ORP value is from one or more individuals known to have suffered a mild stroke, a moderate stroke or a moderately severe stroke, and wherein if the sORP value or icORP of the first sample is significantly lower than the comparable reference value, or if the cORP value is significantly higher than the comparable reference value, determining the individual suffered a severe stroke.

5. The method of claim 1, wherein step a) further comprises measuring the sORP, cORP and/or icORP value of a second sample obtained from the individual some time after obtainment of the first sample and determining if the individual's ORP value has changed over time, and wherein step b) comprises comparing the change in ORP value over time, if any, to a comparable reference value to determine if the individual suffered a severe stroke.

6. The method of claim 5, wherein the time between obtainment of the first sample and obtainment of the second sample is between 6 and 30 hours.

7. The method of claim 5, wherein the reference value is the change in ORP value in the first 24 hours post-stroke stroke in one or more individuals known to have suffered a mild stroke, a moderate stroke or a moderately severe stroke, and wherein if the change, if any, between the sORP value, or icORP value, of the first sample and the sORP value, or icORP value, of the second sample is significantly greater than the comparable reference value, or if the change, if any, between the cORP value of the first sample and the cORP value of the second sample is significantly less than the comparable reference value, determining the individual suffered a severe stroke.

8. A method of determining the likelihood of survival of a stroke patient, comprising:
a) measuring the ORP value of a first sample obtained from the individual, wherein the ORP value is a sORP value, a cORP value and/or a icORP value; and,
b) using the measured ORP value to determine the individual's likelihood of survival.

9. The method of claim 7, wherein the first sample is obtained during initial contact of the patient with a medical professional, or upon admission to a medical facility.

10. The method of claim 8, wherein step b) comprises comparing the measured ORP value of the first sample to a comparable reference ORP value to determine the individual's likelihood of survival.

11. The method of claim 9, wherein the reference ORP value is from one or more individuals known to have survived a stroke, and wherein if the sORP value or icORP of the first sample is significantly lower than the comparable reference value, or if the cORP value is significantly higher than the comparable reference value, determining the individual is unlikely to survive.

12. The method of claim 7, wherein step a) further comprises measuring the sORP, cORP and/or icORP value of a second sample obtained from the individual some time after obtainment of the first sample and determining if the individual's ORP value has changed over time, and wherein step b) comprises comparing the change in ORP value over time, if any, to a comparable reference value to determine the individual's likelihood of survival.

13. The method of claim 12, wherein the time between obtainment of the first sample and obtainment of the second sample is between 6 and 30 hours.

14. The method of claim 12, wherein the reference value is the change in ORP value in the first 24 hours post-stroke in one or more individuals known to have survived a stroke,
and wherein if the change, if any, between the sORP value, or icORP, of the first sample and the sORP value, or icORP value, of the second sample is significantly greater than a comparable reference value, or if the change, if any, between the cORP value of the first sample and the cORP value of the second sample is significantly less than the comparable reference value determining the individual is unlikely to survive.

15. A method for estimating a stroke patients' length of stay in a medical facility, comprising:
a) measuring the ORP value of a first sample obtained from the individual; and,
b) using the measured ORP value to estimate the patient's length of stay in a medical facility.

16. The method of claim 15, wherein step b) comprises comparing the measured OPR value to one or more reference values, wherein the one or more reference values are correlated with the length of stay in the hospital of stroke patients.

17. The method of claim 15, wherein the first sample is obtained during initial contact of the patient with a medical professional, or upon admission to a medical facility.

18. The method of claim 15, wherein the sample is a bodily fluid.

19. The method of any one of 18, wherein the sample selected from the group consisting of blood, plasma, serum and cerebral spinal fluid (CSF).

20. The method of claim 15, wherein the estimated length of stay in the medical facility is used to determine the stroke patients care plan.
